(19) United States
(12) Patent Application Publication
(10) Pub. No.: US 2021/0369690 A1
(43) Pub. Date: Dec. 2, 2021

(54) CONCOMITANT ADMINISTRATION OF GLUCOCORTICOID RECEPTOR MODULATOR RELACORILANT AND CYP2C8 SUBSTRATES

(71) Applicant: Corcept Therapeutics Incorporated, Menlo Park, CA (US)

(72) Inventors: Hazel Hunt, Storrington (GB); Joseph Custodio, Menlo Park, CA (US)

(21) Appl. No.: 17/331,032

(22) Filed: May 26, 2021

Related U.S. Application Data
(60) Provisional application No. 63/030,789, filed on May 27, 2020.

(51) Int. Cl. A61K 31/444; A61K 31/4439; A61K 31/4166

(52) U.S. Cl. CPC A61K 31/444; A61K 31/4166; A61K 31/4439

(57) ABSTRACT

Relacorilant is useful in the treatment of cancer and hypercortisolism. Many drugs useful in treating cancer or hypercortisolism are metabolized by CYP2C8 enzymes. The effects of concomitant administration of relacorilant and a CYP2C8 substrate are disclosed herein.

Relacorilant potently inhibited CYP2C8 in an in vitro test, indicating that co-administration of relacorilant and a CYP2C8 substrate would be expected to increase the CYP2C8 substrate plasma exposure more than five-fold in vivo. Significant reductions in CYP2C8 substrate doses would be expected to be required when administered with relacorilant.

Surprisingly, no such increase in plasma exposure was seen in human studies. Applicant discloses that relacorilant may be safely co-administered with unmodified doses of CYP2C8 substrates such as pioglitazone, rosiglitazone, and enzalutamide. Relacorilant and unmodified doses of enzalutamide may be co-administered to treat cancer, e.g., prostate cancer. Relacorilant and unmodified doses of pioglitazone or rosiglitazone may be co-administered to treat cancer or hypercortisolism.

### CROSS-REFERENCE TO RELATED APPLICATION

This application claims priority to, and the benefit of, U.S. Provisional Patent Application No. 63/030,789, filed May 27, 2020, which application is hereby incorporated by reference herein in its entirety.

### BACKGROUND

The simultaneous, or nearly simultaneous (e.g., concomitant) presence of two drugs in a subject may alter the effects of one or the other, or both, drugs. Such alterations are termed drug-drug interactions (DDIs). For example, the required dose of a drug is often strongly affected by the amount and rate of its degradation in, and elimination from, the body (e.g., by liver or kidney action). However, the presence of a second drug in the body, which is also being acted upon, e.g., by the liver and kidney, can have significant effects on the amount and rate of degradation of the first drug, and can increase or decrease the amount of the first drug that remains in the body at a given time as compared to the amount that would have been present at that time in the absence of the second drug. Thus, for example, the presence of a second drug that is an inhibitor of an enzyme that metabolizes a first drug will inhibit the metabolism of the first drug and thus can often increase the effective dose of the first drug. Where the first drug has toxic side effects, such an increase in effective dose of the first drug may lead to dangerous toxicity that would not have been expected were the second drug not present.

Concomitant administration of different drugs often leads to adverse effects since the metabolism and/or elimination of each drug may reduce or interfere with the metabolism and/or elimination of the other drug(s), thus altering the effective concentrations of those drugs as compared to the effective concentrations of those drugs when administered alone. Thus, concomitant administration of drugs may increase the risk of toxic effects of one or both of the co-administered drugs.

Cytochrome P450 (abbreviated as CYP or P450) enzymes are hemoproteins of approximately 500 amino acids. Fifty-seven human functional CYP genes have been identified. The human CYP genes are classified into 18 families, designated by a Roman numeral, and 44 subfamilies designated by a capital letter. Classification is based on the amino acid sequence identity of the encoded proteins (Nelson, 2009). Eleven enzymes from CYP families 1, 2 and 3 (CYP1A1, CYP1A2, CYP2A6, CYP2B6, CYP2C8, CYP2C9, CYP2C19, CYP2D6, CYP2E1, CYP3A4 and CYP3A5) primarily contribute to drug and chemical metabolism (Guengerich 208; Zanger and Schwab 2013). These enzymes contribute to the biotransformation of approximately 70% of clinically used drugs. Generally, these enzymes provide a clearance mechanism for drugs and other xenobiotics and facilitate elimination from the body in urine and/or bile. CYP represents one of nature's most versatile enzymes with respect to its broad substrate profile and types of biotransformation reactions. The individual CYP enzymes exhibit distinct, but sometimes overlapping, substrate and inhibitor selectivities. Many drugs inhibit the activity of one or more CYP enzymes, and thus have the potential to cause a drug-drug interaction. Thus, a therapeutic dose of a first drug that is metabolized by a CYP enzyme may become a toxic dose when the first drug is administered with a second drug that inhibits that same CYP enzyme, since the CYP enzyme action on the first drug will be reduced by the presence of the second drug, leading to increased levels of the first drug (as compared to the levels obtained by the same dose of the first drug in the absence of the second drug).

Many therapeutically important drugs are metabolized by the CYP2C8 enzyme. CYP2C8 substrate drugs include amodiaquine, cerivastatin, dasabuvir, enzalutamide, imatinib, loperamide, montelukast, paclitaxel, pioglitazone, repaglinide, and rosiglitazone (Beckman et al., Pharmacol Rev 68:168-241 (2016)). For example, the anti-androgen drug enzalutamide (used in treating prostate cancer) is metabolized by CYP2C8; administration to healthy human subjects of the strong CYP2C8 inhibitor gemfibrozil along with enzalutamide more than doubled the amount of enzalutamide and its active metabolite (Gibbons et al., Clin Pharmacokinet (2015) 54:1057-1069). Noting that the recommended dose of enzalutamide was 160 mg/day, these results led Gibbons et al. to state that “[t]o mitigate the risks to patients, it is recommended to reduce the dose of enzalutamide to 80 mg once daily during concomitant use with a strong CYP2C8 inhibitor” (Gibbons et al., page 1067). A similar recommendation was made by Del Re et al.: “Enzalutamide dosage should be therefore reduced in the presence of strong CYP2C8 inhibitors” (Del Re et al., Cancer Treatment Reviews 55 (2017) 71-82, page 78). Del Re et al. concluded that “[t]herefore, caution should be taken when administering combination treatments that may expose the patient to the risk of DDIs” (Del Re et al., page 79).

Relacorilant (see FIG. 1; see also Hunt et al., J. Med. Chem. 60:3405-3421 (2017)) is a selective, non-steroidal modulator of the glucocorticoid receptor that is being investigated in clinical trials in patients with Cushing's syndrome and in patients with various types of cancer including, e.g., prostate cancer.

### SUMMARY

Many therapeutic drugs are substrates of CYP2C8 enzymes; an otherwise safe dose of a first drug metabolized by CYP2C8 may be a toxic dose when concomitantly administered with a second drug that is a CYP2C8 inhibitor. In vitro studies are used to indicate drug combinations expected to suffer from such negative drug-drug interactions (DDIs).

Relacorilant is believed to be useful in treating many disorders, including cancer and hypercortisolism. Relacorilant is further believed to be useful in combination treatments for cancer and in treating hypercortisolism. In vitro tests demonstrated that relacorilant is a potent inhibitor of CYP2C8 (IC₅₀ of 0.21 μM). Such potent inhibition of CYP2C8 would be expected to increase plasma exposure of CYP2C8 substrates by more than five-fold when co-administered with relacorilant. Thus, it was expected that significant reductions in doses of CYP2C8 substrates (e.g., pioglitazone, rosiglitazone, enzalutamide, and others) would be required when administered in combination with relacorilant.

Surprisingly, Applicant determined that it was safe to co-administer relacorilant and a CYP2C8 substrate to human subjects without modifying the dose of the CYP2C8 substrate. Applicant discloses herein that relacorilant may be safely administered along with unmodified doses of pioglitazone, and other CYP2C8 substrates, such as, e.g., rosiglitazone, and enzalutamide. Relacorilant and unmodified doses of enzalutamide may be administered for the treatment of cancer, e.g., prostate cancer. Relacorilant and unmodified doses of pioglitazone or rosiglitazone may be administered for the treatment of cancer, or hypercortisolism.

Accordingly, Applicant discloses herein that a CYP2C8 substrate may be concomitantly administered with the selective glucocorticoid receptor modulator relacorilant without reduction in the dose of the CYP2C8 substrate. Such concomitant administration of a CYP2C8 substrate and relacorilant is believed to be safe for the subject and to provide the therapeutic benefits of both drugs to the subject. In embodiments, the CYP2C8 substrate is pioglitazone, rosiglitazone or enzalutamide.

The methods disclosed herein surprisingly provide safe methods for administering drug combinations that were previously expected to be unsafe, allowing concomitant administration of drug combinations with relacorilant. Such drug combinations are believed to provide more effective treatments than treatment with only one of the drugs in the absence of the other. The surprising ability to safely administer these drug combinations provide advantages including more effective treatments, absence of previously expected side effects, and other advantages.

### BRIEF DESCRIPTION OF THE DRAWING

FIG. 1 shows the chemical structure of relacorilant ((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone).

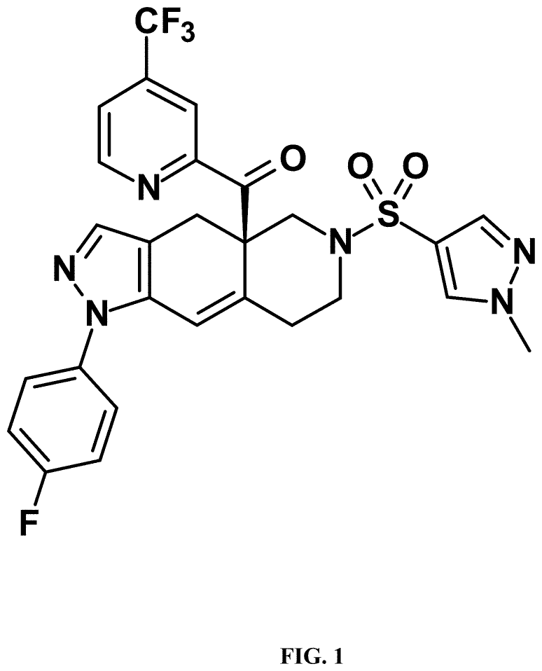

### DETAILED DESCRIPTION

Applicant discloses herein the surprising discovery that relacorilant may be safely co-administered with CYP2C8 substrate drugs without need for reducing the dosage of those CYP2C8 substrate drugs. Such CYP2C8 substrate drugs include enzalutamide, pioglitazone, rosiglitazone, and other CYP2C8 substrates. Relacorilant and a CYP2C8 substrate may be co-administered to treat cancer, such as prostate cancer without need for reducing the dosage of the CYP2C8 substrate. The CYP2C8 substrate drug administered with relacorilant to treat cancer may be, for example, enzalutamide. Relacorilant and a CYP2C8 substrate may be co-administered to treat hypercortisolism, e.g., to treat Cushing's syndrome and Cushing's Disease without need for reducing the dosage of the CYP2C8 substrate. The CYP2C8 substrate drug administered with relacorilant to treat hypercortisolism may be, for example, pioglitazone or rosiglitazone. Such co-administration of relacorilant and a CYP2C8 substrate provides therapeutically effective levels of both relacorilant and of the CYP2C8 substrate at the same time in the patient.

In embodiments, Applicant discloses a method of treating a disorder, comprising administering to a patient in need of treatment for said disorder:

a) an effective dose of relacorilant; and

b) an effective dose of a therapeutic agent, wherein said therapeutic agent is a substrate for CYP2C8 enzyme metabolism, said therapeutic agent having a single agent dose when administered without other pharmaceutical agents, wherein said therapeutic agent effective dose is substantially the same as said single agent dose;

Wherein a) and b) are performed at times effective to provide the patient with an effective level of relacorilant and an effective level of the therapeutic agent at the same time,

Whereby the disorder is treated.

In embodiments, the therapeutic agent may be rosiglitazone, pioglitazone, or enzalutamide. In embodiments, the disorder is cancer, and may be prostate cancer. In embodiments, the therapeutic agent is an antiandrogen, and may be enzalutamide. In embodiments, the disorder is hypercortisolism. In embodiments, the therapeutic agent is rosiglitazone or pioglitazone.

For example, applicant has surprisingly discovered that relacorilant may be administered to subjects concomitantly receiving enzalutamide without the need to make dose modifications due to CYP2C8 inhibition. This discovery is surprising, since relacorilant has been shown to be a potent inhibitor of CYP2C8 in vitro and enzalutamide is predominantly metabolized by CYP2C8. However, in a clinical study in healthy volunteers designed to assess the propensity for relacorilant to cause a drug-drug interaction with the CYP2C8 substrate pioglitazone, the expected increase in pioglitazone concentration was not observed, indicating that relacorilant does not inhibit CYP2C8 in a clinical setting.

Applicant discloses herein that relacorilant may be safely administered along with unmodified doses of CYP2C8 substrates. Applicant discloses herein that relacorilant may be safely administered along with unmodified doses of CYP2C8 substrates such as, e.g., pioglitazone, rosiglitazone, enzalutamide, amodiaquine, cerivastatin, dasabuvir, imatinib, loperamide, montelukast, paclitaxel, and repaglinide.

Applicant's surprising discovery is believed to apply to patients suffering from a disease or disorder and receiving a drug metabolized by CYP2C8. For example, patients receiving pioglitazone for the treatment of a disorder, such as hypercortisolism, may benefit from concomitant treatment with pioglitazone and relacorilant, and may continue to receive pioglitazone at its therapeutic dose without need for reducing the dose of pioglitazone. Similarly, patients receiving rosiglitazone for the treatment of a disorder, such as hypercortisolism, may benefit from concomitant treatment with rosiglitazone and relacorilant, and may continue to receive rosiglitazone at its therapeutic dose without need for reducing the dose of rosiglitazone.

Applicant's surprising discovery is believed to apply to patients suffering from a disease or disorder and receiving a drug metabolized by CYP2C8. For example, patients receiving enzalutamide for the treatment of cancer, such as prostate cancer, may benefit from concomitant treatment with enzalutamide and relacorilant, and may continue to receive enzalutamide at its therapeutic dose without need for reducing the dose of enzalutamide.

In embodiments, relacorilant is administered orally. In embodiments, relacorilant, is administered on a daily basis; for example, in embodiments, relacorilant is administered once per day. In embodiments, relacorilant is administered with food. Administered “with food” means that the patient has begun eating a meal within 30 minutes, or within one hour, of the time that relacorilant is administered. For example, relacorilant may be administered to a patient with a meal, or soon after (e.g., within half an hour) the patient began eating the meal.

In alternative embodiments, relacorilant is administered to a fasted patient, i.e., to a patient who has not eaten food for at least one hour, or at least two hours, or more hours prior to relacorilant administration. For example, relacorilant may be administered to a fasted patient in the morning, i.e., to a patient who has not yet eaten the morning meal, and has not eaten since the evening meal of the prior evening.

In embodiments, relacorilant is administered daily, at a daily dose of relacorilant of between about 1 and 100 mg/kg/day, preferably a daily dose of relacorilant of between about 1 and 20 mg/kg/day. In embodiments, the daily dose of relacorilant is between about 10 and about 2000 milligrams (mg), or between about 50 and about 1500 mg, or between about 100 and about 1000 mg relacorilant. In embodiments, a daily dose of relacorilant may be about 10 mg, or 15 mg, or 20 mg, or 25 mg, or 50 mg, or 100 mg, or 150 mg, or 200 mg, or 250 mg, or 300 mg, or 350 mg, or 400 mg, or 450 mg, or 500 mg, or 550 mg, or 600 mg, or 650 mg, or 700 mg, or 750 mg, of 800 mg, or 850 mg, or 900 mg, or 950 mg of relacorilant. In embodiments, an effective dose of relacorilant is between 75 milligrams per day (mg/day) and 200 mg/day, and may be selected from 75 mg/day, 100 mg/day, 125 mg/day, 150 mg/day, 175 mg/day, and 200 mg per day. In embodiments, the effective dose of relacorilant is 100 mg/day, 125 mg/day, or 150 mg/day. In embodiments, an effective relacorilant dose for treatment of cancer is between about 75 mg/day and about 200 mg/day, and may be, e.g., 100 mg/day, or 125 mg/day, or 150 mg/day. In embodiments, an effective relacorilant dose for treatment of hypercortisolism or a disorder associated with hypercortisolism is between about 50 mg/day and about 500 mg/day, and may be, e.g., 150 mg/day, or 200 mg/day, or 250 mg/day, or 300 mg/day, or 350 mg/day, or 400 mg/day. In embodiments, the relacorilant dose may be adjusted (e.g., increased) from an initial dose during the course of treatment.

### Definitions

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. Administration may be by oral administration (i.e., the subject receives the compound or composition via the mouth, as a pill, capsule, liquid, or in other form suitable for administration via the mouth). Oral administration typically involves swallowing the pill, capsule, liquid, or other formulation. Oral administration may include buccal administration (where the compound or composition is held in the mouth, e.g., under the tongue, and absorbed there).

Other examples of modes of administration include, e.g., by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramuscular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art.

As used herein, the terms “co-administration”, “concomitant administration”, “combined administration”, “combination treatment”, and the like refer to the administration of at least two pharmaceutical agents to a subject to treat a disease or condition. The two agents may be administered simultaneously, or sequentially in any order during the entire or portions of the treatment period. The at least two agents may be administered following the same or different dosing regimens. Such agents may include, for example, e.g., relacorilant and another drug, which may be, e.g., a drug useful in treating hypercortisolism, may be a drug useful in treating cancer, or another therapeutic agent. In some cases, one agent is administered following a scheduled regimen while the other agent is administered intermittently. In some cases, both agents are administered intermittently. In some embodiments, the one pharmaceutical agent may be administered daily, and the other pharmaceutical agent may be administered every two, three, or four days.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Therapeutic agents such as relacorilant, pioglitazone, rosiglitazone, enzalutamide, and others, are typically administered in capsules, tablets, or other formulations which include the active agent and one or more pharmaceutically acceptable carriers. The use of such media and agents for pharmaceutically active substances is well known in the art. Except insofar as any conventional media or agent is incompatible with the active compound, use thereof in the compositions is contemplated. Supplementary active agents can also be incorporated into the compositions.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

Relacorilant (((R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone)) is a GRM. Relacorilant is described in Example 18 of U.S. Pat. No. 8,859,774 (hereby incorporated by reference).

As used herein, the term “CYP2C8” refers to the cytochrome P450 enzyme subtype 2C8. In humans, the most common form has 490 amino acids, and has the UniProtKB accession number P10632.2. The gene encoding CYP2C8 has Gene ID 1558.

CYP2C8 substrate drugs include amodiaquine, cerivastatin, dasabuvir, enzalutamide, imatinib, loperamide, montelukast, paclitaxel, pioglitazone, repaglinide, and rosiglitazone (Beckman et al., Pharmacol Rev 68:168-241 (2016)).

### Example 1. In Vitro CYP Inhibition Assay

Cytochrome P450 (CYP) isoforms CYP2B6, CYP2C8 and CYP3A5, heterologously expressed in E. coli, were obtained from Cypex and mixed to produce a 3-CYP mix. A selective and FDA accepted substrate for each isoform was present in the reaction at a concentration around its Kₘ.

Relacorilant (final concentration range 0.032-10 μM, 1% DMSO) or a cocktail of control CYP inhibitors was added to reaction tubes in a 96 well plate format. The 3-CYP mix and a CYP substrate cocktail were added and the tubes warmed for 3 minutes whilst mixing on a BioShake IQ (37° C., 1500 rpm). NADPH (final concentration 1 mM) was added and the mixture was incubated for 10 minutes. Methanol containing an internal standard (1 μM tolbutamide) was then added to all samples, and these were mixed and placed at −20° C. for ≥1 hour to quench the reaction and allow protein to precipitate.

All samples were centrifuged (2500×g, 20 minutes, 4° C.). The supernatants were transferred to a fresh 96 well plate, compatible with an autosampler. The plate was sealed with a pre-slit silicone mat and the metabolites were analyzed by LC-MS/MS.

Control CYP inhibitors (IC₅₀—appropriate concentration range, final assay concentration 1% DMSO) were added as a cocktail: CYP2B6, ticlopidine; CYP2C8, quercetin; CYP3A5, ketoconazole.

The final concentration in the assay of the 3-CYP mix was 18 pmol/mL for CYP2B6, 1 pmol/mL for CYP2C and 5 pmol/mL for CYP3A5.

The CYP substrate cocktail comprised the following components: CYP2B6, bupropion; CYP2C8, amodiaquine; CYP3A5, midazolam. The solvent was methanol for all stock solutions and the final concentration of methanol in the assay was 0.625%.

The metabolites measured were: CYP2B6, hydroxybupropion; CYP2C8, N-desethyl amodiaquine; CYP3A5, 1′-hydroxymidazolam.

All reactions were performed in duplicate at 37° C. and in 0.1 M phosphate buffer (pH 7.4). The final protein concentration was 0.06 mg/ml.

### Data Processing

Data were processed and the results reported as an IC₅₀ value (concentration resulting in a 50% inhibition of response), generated from a pseudo-Hill plot, the slope and y axis intercept being used to calculate the IC₅₀ according to the following equation.

$${IC}_{50} = 1 {0}^{\frac{intercept}{slope}}$$

Relacorilant inhibited CYP2C8 with a mean IC₅₀ value of 0.21 μM in this assay.

Based on the in vitro data showing that relacorilant potently inhibited CYP2C8 with a mean IC₅₀ value of 0.21 co-administration of a therapeutic concentration of relacorilant with a CYP28 substrate would be expected to result in a greater than 5-fold increase in the plasma exposure of the CYP2C8 substrate, relative to administration of the CYP2C8 substrate alone.

### Example 2. Clinical Drug-Drug Interaction Study in Healthy Volunteers

The results of the study described in Example 1 indicated that co-administration of relacorilant and a CYP2C8 substrate to a human subject would lead to large increases in plasma exposure of the CYP2C8 substrate as compared to that CYP2C8 substrate's plasma exposure in the absence of relacorilant.

An open-label, crossover study was conducted in healthy subjects to determine the effect of relacorilant on the plasma exposure of pioglitazone, a known probe substrate CYP2C8. A single dose of 15 mg of pioglitazone was administered alone and pharmacokinetic (PK) samples were collected before dosing (0 hour) and at 0.5, 1, 1.5, 2, 2.5, 3, 4, 6, 8, 12, 18, 24, 36, 48, 60, and 72 hours post-dose. Relacorilant (350 mg) was then administered once a day for 10 consecutive days. On the following day, a single dose of 15 mg of pioglitazone was administered in combination with relacorilant 350 mg and pharmacokinetic (PK) samples were again collected at pre-dose through 72 hours post-dose at the same timepoints as described above. The plasma concentrations of pioglitazone and its metabolite, pioglitazone M4 were evaluated by validated bioanalytical assays on each dosing occasion of pioglitazone. The PK results showed that once-daily dosing of relacorilant did not increase the plasma exposures of pioglitazone or its metabolite, indicating a lack of an inhibitory effect of relacorilant on CYP2C8 (Table 1). Although CYP2C8 inhibition by relacorilant had been previously observed in vitro, the surprising results of the clinical drug interaction study demonstrated that relacorilant does not inhibit CYP2C8 in vivo.

TABLE 1
Statistical Comparisons of Plasma Pioglitazone and its Metabolite
Pharmacokinetic Parameters: Day 15 (Treatment E) vs Day 2
(Treatment B) (PK Population)

| | Test | | Reference | | | |
|---|---|---|---|---|---|---|
| | (Day 15) | | (Day 2) | | | |
| | Treatment E | | Treatment B | | Ratio of | 90% |
| Parameter | Geometric | | Geometric | | Geometric | Confidence |
| (unit) | LSM | n | LSM | N | LSMs (%) | Intervals |

Pioglitazone

| | | | | | | |
|---|---|---|---|---|---|---|
| Cₘₐₓ (ng/mL) | 376.5 | 26 | 483.8 | 27 | 77.82 | 69.65-86.96 |
| AUC_{0-tz} | 3953 | 26 | 5290 | 27 | 74.71 | 68.06-82.02 |
| (ng · h/mL) | | | | | | |
| AUC_{inf} | 4047 | 25 | 5408 | 27 | 74.83 | 68.11-82.21 |
| (ng · h/mL) | | | | | | |

Pioglitazone M4

| | | | | | | |
|---|---|---|---|---|---|---|
| Cₘₐₓ (ng/mL) | 253.9 | 26 | 237.3 | 27 | 106.99 | 99.70-114.81 |
| AUC_{0-tz} | 10460 | 26 | 10460 | 27 | 99.97 | 94.80-105.43 |
| (ng · h/mL) | | | | | | |
| AUC_{inf} | 12590 | 25 | 12890 | 26 | 97.68 | 92.98-102.62 |
| (ng · h/mL) | | | | | | |

ANOVA, analysis of variance;
AUC_{inf}, AUC from time 0 extrapolated to infinity;
AUC_{0-tz}, AUC from time 0 until the time of the last measurable concentration;
Cₘₐₓ, maximum plasma concentration;
CV %, coefficient of variation;
LSM, least squares mean.
Treatment B: Single oral dose of 15 mg of pioglitazone hydrochloride (Reference).
Treatment E: Single oral dose of 15 mg of pioglitazone hydrochloride and 350 mg relacorilant administered on Day 15 followed by oral doses of 350 mg relacorilant administered QD on Days 16 and 17 (Test).
Parameters were ln-transformed prior to analysis.
Geometric LSMs were calculated by exponentiating the LSMs from the ANOVA.
Ratio of Geometric LSMs = 100*(Test/Reference); where Test is Treatment E and Reference is Treatment B.

All patents, patent publications, publications, and patent applications cited in this specification are hereby incorporated by reference herein in their entireties as if each individual publication or patent application were specifically and individually indicated to be incorporated by reference. In addition, although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating a disorder, the method comprising administering to a patient in need of treatment for said disorder:
a) an effective dose of relacorilant; and
b) an effective dose of a therapeutic agent, wherein said therapeutic agent is a substrate for CYP2C8 enzyme metabolism, said therapeutic agent having a single agent dose when administered without other pharmaceutical agents, wherein said therapeutic agent effective dose is substantially the same as said single agent dose;
Wherein a) and b) are performed at times effective to provide the patient with an effective level of relacorilant and an effective level of the therapeutic agent at the same time,
Whereby the disorder is treated.

2. The method of claim 1, wherein the relacorilant dose is between about 1 milligram per kilogram (mg/kg) and about 20 mg/kg.

3. The method of claim 1, wherein the relacorilant dose is between about 25 mg and about 500 mg.

4. The method of claim 4, wherein the relacorilant dose is administered at a time within about 15 minutes of the time of administration of the therapeutic agent dose.

5. The method of claim 1, wherein the relacorilant dose and therapeutic agent dose are administered sequentially, wherein said sequential administration is selected from a) the relacorilant dose is administered to the patient, and then, after a time interval, the therapeutic agent dose is administered to the patient, and b) the therapeutic agent dose is administered to the patient, and then, after a time interval, the relacorilant dose is administered to the patient.

6. The method of claim 5, wherein said time interval is selected from about one hour and about one day.

7. The method of claim 1, wherein the therapeutic agent is selected from rosiglitazone, pioglitazone, and enzalutamide.

8. The method of claim 1, wherein said disorder is cancer.

9. The method of claim 8, wherein said disorder is prostate cancer, and the therapeutic agent is enzalutamide.

10. The method of claim 1, wherein said disorder is hypercortisolism, and the therapeutic agent is selected from rosiglitazone and pioglitazone.

11. A method of treating cancer, the method comprising administering to a patient in need of cancer treatment:
a) relacorilant, and
b) an anticancer agent, wherein said anticancer agent is a substrate for CYP2C8 enzymes, the anticancer agent having a therapeutic dose when it is administered to a subject in the absence of other pharmaceutical agents, and wherein said anticancer agent is administered to the patient at a dose that is substantially the same as said therapeutic dose.

12. The method of claim 11, wherein said relacorilant is administered to said patient at a dosage of between about 1 milligram per kilogram (mg/kg) and about 20 mg/kg.

13. The method of claim 11, wherein said relacorilant is administered to said patient at a dose of between about 25 mg and about 500 mg per day.

14. The method of claim 11, wherein said relacorilant is administered to said patient at a dose of between about 75 mg and about 200 mg per day.

15. The method of claim 11, wherein said relacorilant dose is administered to the patient at a time within about 15 minutes of the time of administration of the anticancer agent.

16. The method of claim 11, wherein said relacorilant and said anticancer agent are administered sequentially to the patient, wherein said sequential administration is selected from a) the relacorilant dose is administered to the patient, and then, after a time interval, the anticancer agent is administered to the patient, and b) the anticancer agent is administered to the patient, and then, after a time interval, the relacorilant dose is administered to the patient.

17. The method of claim 16, wherein said time interval is selected from about one hour and about one day.

18. The concomitant use of relacorilant and a CYP2C8 substrate in the treatment of a disorder, wherein CYP2C8 substrate alone has been used in treating said disorder, wherein the amount of said CYP2C8 substrate used in said concomitant treatment with relacorilant is the same amount of CYP2C8 substrate as used alone in treating said disorder.

19. The use of claim 18, wherein the amount of relacorilant is between about 25 mg and about 1500 mg.

20. The use of claim 19, wherein a) said disorder is hypercortisolism, and the CYP2C8 substrate is selected from rosiglitazone and pioglitazone, or b) said disorder is prostate cancer and w the CYP2C8 substrate is enzalutamide.
